(19) United States
(12) Patent Application Publication
(10) Pub. No.: US 2023/0180749 A1
(43) Pub. Date: Jun. 15, 2023

(54) Stable Botanical Antimicrobial Compositions

(71) Applicant: The Trustees of Columbia University in the City of New York, New York, NY (US)

(72) Inventors: SHANTA M. MODAK, RIVER EDGE, NJ (US); NAYANA BAIJU, Kochi (IN); CHATHURANGA C. DE SILVA, CLIFFSIDE PARK, NJ (US)

(21) Appl. No.: 18/092,899

(22) Filed: Jan. 3, 2023

Related U.S. Application Data
(63) Continuation of application No. PCT/US2021/040271, filed on Jul. 2, 2021.
(60) Provisional application No. 63/047,493, filed on Jul. 2, 2020.

(51) Int. Cl. A01N 37/06; A01N 31/08; A01N 65/22; A01N 37/10; A01N 37/36; A01P 1/00; A01N 65/44; A01N 65/36; A01N 65/24; A01N 65/08; A61Q 19/00; A61K 8/9789; A61K 8/36; A61K 8/368; A23L 3/3463; A23L 3/3508

(52) U.S. Cl. CPC A01N 37/06; A01N 31/08; A01N 37/10; A01N 37/36; A01N 65/08; A01N 65/22; A01N 65/24; A01N 65/36; A01N 65/44; A01P 1/00; A23L 3/3508; A23L 3/34635; A61K 8/361; A61K 8/368; A61K 8/9789; A61Q 19/007; A61K 2800/524

(57) ABSTRACT

The present technology relates to botanical antimicrobial compositions that comprise various ingredients, including undecylenic acid, organic acid or solvents; and that can be used as surface cleaners, disinfectants, sanitizers, or preservatives for food or consumer products. The present technology also relates to methods of disinfecting or sanitizing surfaces, or preserving food or personal care compositions, comprising contacting such surfaces, or food or personal care compositions, with an antimicrobial composition herein.

### CROSS-REFERENCE TO RELATED APPLICATIONS

This application is a Continuation of International Application Number PCT/US21/40271, filed on Jul. 2, 2021, which claims the benefit of priority to U.S. Provisional Application No. 63/047,493, filed on Jul. 2, 2020, the disclosure of each is hereby incorporated by reference in its entirety.

### BACKGROUND

The present technology relates to antimicrobial compositions, particularly those suitable for disinfecting surfaces, and acting as food or preservative agents.

Microbes live in every part of the biosphere including air, water, and soil. Pathogenic microbes can cause infection and other complications in public, hospital, institutional, business and home settings. Although not all microbes are pathogenic, many microbes produce by-products that are aesthetically displeasing, such as foul odors and discoloration. Further, personal care products including wound care products, lotions and cosmetics can deteriorate if exposed to microbes. Antimicrobial compositions that can be used for a variety of applications, including sanitizing of surfaces and preservation of food and cosmetics, are always desirable.

An ongoing need exists for antimicrobial surface sanitizing and preservative compositions that are fast acting and effective, that can adequately clean surfaces and prolong the shelf life of foods and usefulness of consumer products, that contain natural, non-irritating ingredients, and that exhibit prolonged stability.

### SUMMARY

In certain embodiments, the present technology is directed to a botanical antimicrobial composition comprising:
(a) 0.01 to 5% undecylenic acid; or 0.01 to 5% thymol; or a combination thereof;
(b) one or more solvents selected from ethanol, 1,3 propanediol, or any combination thereof; and
(c) an organic acid, wherein the organic acid is benzoic acid, citric acid, lactic acid, salicylic acid, mandelic acid, maleic acid or any combination thereof.

In certain embodiments, the present technology is directed to a botanical antimicrobial composition comprising:
(a) 0.01 to 2% undecylenic acid; or 0.01 to 1% thymol; or a combination thereof;
(b) 1 to 80% of one or more solvents selected from ethanol, 1,3 propanediol, or any combination thereof; and
(c) 0.1 to 20% organic acid, wherein the organic acid is benzoic acid, citric acid, lactic acid, salicylic acid, mandelic acid, maleic acid, or any combination thereof.

In certain embodiments, the present technology is directed to a composition that further comprises one or more of the following:
(d) an alkanediol selected from pentylene glycol, octanediol or decanediol;
(e) 0.02 to 5% of one or more essential oils selected from lemongrass oil, lemon oil, orange oil, cinnamon oil, cedarwood oil, myrrh oil, geranium oil or bergamot oil; or
(f) one or more surfactants or chelating agents selected from sodium dodecyl sulfate, cocoamidopropyl betaine, cocoamidopropylamine oxide (Incromine oxide), cetrimonium chloride, ethylenediaminetetraacetic acid (EDTA), or any combination thereof.

In certain embodiments, a composition herein can further comprise one or more of the following:
(d) 0.1 to 20% solvent or emollient solvent selected from glycerin, propylene glycol, 1,3 propanediol, benzyl alcohol, phenoxyethanol, phenylethanol, isopropanol, or any combination thereof;
(e) 1 to 20% alkanediol selected from pentylene glycol, octanediol, or decanediol;
(f) one or more essential oils or extracts selected from wasabi extract, cinnamon bark oil, willow bark oil, lemongrass oil, lemon oil, orange oil, cinnamon oil, cedarwood oil, myrrh oil, geranium oil, bergamot oil, or any combination thereof; and
(g) 0.01 to 5% surfactant or chelating agent selected from sodium dodecyl sulfate, Cocoamidopropyl betaine, Incromine oxide, cetrimonium chloride, EDTA or any combination thereof.

In certain embodiments, the present technology is directed to a preservative composition comprising:
(a) 30 to 50% undecylenic acid; or 1 to 5% thymol, or a combination thereof;
(b) 5 to 50% of one or more solvents selected from ethanol, propanediol, or any combination thereof; and
(c) 10 to 20% organic acid, wherein the organic acid is benzoic acid, citric acid, lactic acid, salicylic acid, mandelic acid, maleic acid, or any combination thereof.

In certain embodiments, the present technology is directed to surface disinfecting or sanitizing compositions, food, or personal care preservative compositions comprising the antimicrobial compositions herein.

In certain embodiments, the present technology is directed to methods of sanitizing or disinfecting a surface, or methods of preserving food or personal care compositions, comprising contacting a composition herein with the surface, food or personal care composition.

### DETAILED DESCRIPTION

All ingredient and formulation percentages expressed herein are by weight (w/w) unless explicitly noted in specific formulations. All ingredients are at 100% purity/concentration unless otherwise noted.

As used herein, “sanitizing” or “sanitizer” are used interchangeably and refer to the preservation of health by removing, preventing, neutralizing, or controlling any agent that injures the health, such as a microbe.

As used herein, “disinfecting” or “disinfectant” are used interchangeably and refer to the destruction or inhibition of growth or effect of any biological agent or contaminant, such as a microbe. Generally, disinfectants are used primarily for surfaces.

As used herein, “antimicrobial” or “antimicrobial agent” means an agent that kills microorganisms or stops their growth. These include, but are not limited to, antibacterial agents, antifungal agents, antiviral agents, microbiocidal agents, antibiotics, bactericidal agents, bacteriostatic agents, disinfectants, sanitizers, and antiseptics. Generally, antiseptics are used primarily for living tissue and skin.

In certain embodiments, the compositions herein are stable. As used herein, “stable” means exhibiting efficacy after more than 1 year. As used herein, “exhibiting efficacy” means showing more than 4.0 log₁₀ reduction in bacterial growth from that of control in the EN1276 test when challenged with 5 × 10⁸ CFU of bacteria, within 5 minutes of contact. In certain embodiments, the 1 year period is achieved through simulating such conditions with the accelerated aging method. The accelerated aging method can be conducted by keeping the solutions at 45° C. for 8 weeks, which is equivalent to stability of 1 year under regular, room temperature conditions.

In certain embodiments, a composition herein can include an antimicrobial composition having long term stability and completely or substantially all natural ingredients, including botanical ingredients. For example, a composition herein can be a completely or substantially completely natural surface disinfectant, antimicrobial, food, or preservative composition.

In certain embodiments, a composition herein comprises an antibacterial agent, wherein the antibacterial agent is undecylenic acid, thymol or a combination thereof.

In certain embodiments, a composition herein comprises 0.01 to 5% undecylenic acid, or 0.01 to 2% undecylenic acid, 0.02 to 5% thymol, or any combination thereof.

In certain embodiments, a composition herein further comprises any of the following:
1 to 80% or 1 to 20% or 0.1 to 20% 1,3 propanediol;
0.1 to 15% of an organic acid, for example, benzoic, citric, lactic, salicylic, mandelic, or maleic acid or any combination thereof;
alkanediol, for example, pentylene glycol, octanediol or decanediol;
0.02 to 5% essential oil, for example, lemongrass oil, lemon oil, orange oil, cinnamon oil, cedarwood oil, myrrh oil, geranium oil or bergamot oil;
0.2 to 40% or 0.01 to 1% of one or more surfactants, emollients, solvents, alcohols or chelating agents, for example, sodium dodecyl sulfate, glycerin, propylene glycol, benzyl alcohol, phenoxyethanol, phenyl ethanol, alcohol ethanol, isopropanol, cetrimonium chloride, sodium phytate, ethylenediaminetetraacetic acid (EDTA), or any combination thereof;

In certain embodiments, the compositions herein are useful as surface disinfectants. As used herein, “surface” refers to any part of any item that may come into contact with users and that can be desired to be cleaned or sanitized. In certain embodiments, the compositions herein are useful as preservatives for food or consumer products such as personal care products.

As used herein, “food” refers to any substance that is ingested for nutrition or growth.

As used herein, “personal care product” or “personal care composition” refers to a consumer product that consumers use for personal hygiene or beautification. These include, for example, cosmetics, lotions, hair care products, and personal cleansing or grooming products for any part of the body. In certain embodiments herein, a personal care product can comprise a ratio of an antimicrobial composition herein in a personal care product carrier.

As used herein, a “personal care product carrier” means any substance suitable for applying to the body of a user (e.g., the skin or hair) for the benefit of the user, and that includes a personal care product or personal care composition. Examples of suitable personal care product carriers include, e.g., a cream, a gel, a salve, a paste, a spray, a foam, a liquid, a suspension, a powder, or a solid. In certain embodiments, the present technology is directed to a composition (for example, a preservative composition) comprising 1 to 2% of any composition described herein, in a personal care product carrier - that is, for example, 1 to 2 grams of the antimicrobial composition mixed in with 100 grams of the personal care product carrier.

In certain embodiments, the products herein can significantly reduce bacteria counts in as little as five minutes of contact with bacteria. Furthermore, in certain embodiments, the compositions herein are at least partly or entirely biodegradable, thus making it useful for consumer use in a variety of applications, including personal care products (for example, cosmetics, hand sanitizers, cleansers, lotions), food, and surface disinfectants.

In certain embodiments, the products herein can significantly reduce bacteria counts in as little as five minutes of contact with bacteria before, during or after a detergent wash. Furthermore, in certain embodiments, the compositions herein are at least partly or entirely biodegradable.

As used herein, “essentially free of” an ingredient means containing less than 5% of such ingredient. As used herein, “completely free of” an ingredient means containing none of that ingredient. In various embodiments, the compositions herein contain less than 5%, less than 2%, or less than 1% or 0% of non-plant derived sanitizing agents, including those commonly used sanitizing agents mentioned herein.

In certain embodiments, the compositions herein are essentially free of, or completely free of, any synthetic sanitizing agents. As used herein, “synthetic sanitizing agent,” “synthetic sanitizer,” “synthetic antimicrobial” or “synthetic antibacterial” all mean a sanitizing, antimicrobial or antibacterial agent derived from non-natural ingredients, including petroleum products. In certain embodiments, such non-botanical or synthetic sanitizing or antimicrobial agents can include any of the following ingredients: quaternary ammonium compounds (QAC), phenolics (Triclosan®, Parachlorometaxylenol (PCMX)), harsh chemicals such as sodium hypochlorite (bleach), sodium hydroxide, hydrogen peroxide, and compounds containing ammonia, chlorine, or perchloroethylene, all of which are well-known irritants.

In certain embodiments, the compositions herein, even when subject to high dilution (for example, up to and including 60-fold dilution), still achieve antimicrobial efficacy and keep bacteria counts low during EN1276 method (the European Standard for evaluation of for evaluation of bactericidal efficacy of disinfecting liquids) and EN 13697 (Quantitative non-porous surface test for the evaluation of bactericidal and/or fungal activity of chemical disinfectants used in food, industrial, domestic and institutional areas) testing in both clean and dirty conditions. For example, in certain embodiments, a composition herein can achieve at least 3.0 log₁₀ reduction, or at least 5.0 log₁₀ reduction, in bacterial growth from that of control when challenged with 1 × 10⁷ to 5 × 10⁸ CFU of bacteria during, or within (up to) 5 minutes of contact.

Another aspect of certain embodiments of the present technology is the addition of one or more solvents, including but not limited to: ethanol, benzyl alcohol (commonly found in fruits and teas and also found in a variety of essential oils, including jasmine and hyacinth), phenoxyethanol (derived from grapefruit), or 1,3-propanediol (for example, that derived from corn sugar and available under the trade name Zemea® from Dupont and Lyle Bioproducts, Loudon, TN, USA). In certain embodiments, the one or more solvents are included in amounts of 30 to 60% of the compositions herein, and can serve to enhance the antimicrobial efficacy. In certain embodiments, the compositions herein can achieve a maximum of 5.6 log₁₀ reduction in bacterial growth from that of control when challenged with 1 × 10⁷ to 5 × 10⁸ CFU of bacteria during, or within (up to) 5 minutes of contact.

In certain embodiments, the surface disinfectant or sanitizing compositions, preservative compositions, personal care compositions, or food compositions herein are completely or substantially completely natural - that is, they are completely or substantially derived from naturally-occurring sources, rather than synthetic sources. In certain embodiments, the technology herein is directed to compositions that include sanitizers that are botanical. As used herein, “botanical” means obtained from a plant. Thus, in certain embodiments, the compositions herein mainly contain ingredients sourced from plants; and when referring herein to “botanical antimicrobial compositions” it is meant that such compositions contain one or more botanical ingredients.

As used herein, “essentially botanical” means at least 95% from botanical sources. In certain embodiments, the compositions herein include sanitizing agents that are essentially botanical. In certain embodiments, the compositions herein are essentially free of, or completely free of, non-botanical sanitizing agents. As used herein, “non-botanical” means not derived from plants.

As used herein, “essential oil” (EO) is a volatile oil obtained from a plant or an animal source that comprises one or more active agent (also referred to herein as an Isolated Component or “IC” or “constituent” or “ingredient”) which may be, for example but not by way of limitation, a monoterpene or sesquiterpene hydrocarbon, alcohol, ester, ether, aldehyde, ketone, or oxide. Essential oils are commonly extracted by distillation, expression, extraction, resin tapping, wax embedding or cold pressing. Isolated components generally fall into the following categories: acids, alcohols (e.g., monoterpenols or sesquiterpenols), aldehydes, coumarins, esters, ketones, lactones, terpenes (e.g., monoterpenes or sesquiterpenes), oxides, or phenols.

As used herein, “botanical extract” means a composition from a plant source (a botanical) that is prepared by soaking the botanical in a solvent (such as water or alcohol). A botanical extract refers to the resultant liquid, which contains the essential oil with the solvent. As used herein, “extract” generally means any composition prepared in this manner, whether or not it is from a botanical source.

For example, “thyme oil” refers to essential oil produced through methods such as steam distillation of the leaves and flowers of the thyme plant. Thyme oil contains organic compounds such as p-cymene, myrcene, borneol, carvacrol, linalool and thymol. Thyme oil generally contains 20 to 54% thymol. When “thymol/thyme oil” is mentioned here, this refers generally to any materials derived from the thyme plant, and can mean either thymol, thyme oil or a combination of both.

### Undecylenic Acid

In various embodiments, antimicrobial compositions herein comprise undecylenic acid. Undecylenic acid is an unsaturated fatty acid typically used as an antifungal on the skin. Although the antimicrobial activity of undecylenic acid was previously known to be “restricted to the inhibition of moulds including pathogenic fungi” (W. Paulus, Microbicides for the Protection of Materials: A Handbook, p. 209), the present embodiments are directed to the surprising finding that undecylenic acid and organic acid demonstrate synergistic efficacy in killing bacteria. In various embodiments, an antimicrobial composition herein comprises 0.01 to 5%, or 0.01 to 2%, or 0.1 to 0.5%, or 0.3 to 6%, or 10 to 50% undecylenic acid.

In certain embodiments, the compositions herein exhibit prolonged stability by employing specific solvent mixtures in the formulations. In certain embodiments, the present technology is directed to disinfecting or preservative compositions comprising undecylenic acid, organic acid and or a salt thereof, ethanol; and in certain embodiments can further comprise any of the following: alkanediol (e.g., 1,3-propanediol and octanediol), propylene glycol, or sodium dodecyl sulfate.

Certain useful and desirable properties of the compositions herein can be illustrated in the following Examples.

### Example 1

### Surface Disinfectant Formulations and Efficacy

Surface disinfectant compositions were prepared, comprising the following ingredients:
(a) 0.01 to 5% undecylenic acid, 0.01 to 5% thymol or any combination thereof;
(b) 1 to 80%, ethanol, propanediol or any combination thereof;
(c) 0.1 to 15% organic acid selected from benzoic, citric, lactic acid or any combination thereof;
(d) alkanediol selected from pentylene glycol, octanediol or decanediol;
(e) 0.01 to 5% essential oil selected from lemongrass oil, lemon oil, orange oil, cinnamon oil, cedarwood oil, myrrh oil, geranium oil, bergamot oil or any combination thereof;
(f) 0.01 to 5% surfactant or chelating agents selected from sodium dodecyl sulfate, cocoamidopropyl betaine, incromine oxide, cetrimonium chloride, EDTA or any combination thereof; and
(g) 0.1 to 20% solvent or emollient solvent selected from glycerin, propylene glycol, benzyl alcohol, phenoxyethanol, phenylethanol, ethanol, isopropanol or any combination thereof.

The following surface disinfectant formulations were prepared and tested for their antibacterial efficacy by EN 1276 and EN 13697 method against S. aureus. The efficacy is expressed as log reduction from the control group. Both composition and efficacy (log reduction) of surface disinfectants are given below.

Study A. Direct Use Surface Disinfectant Formulations (Spray or Wipes)

TABLE 1

Main Ingredient Ranges

| Ingredient | %w/w |
|---|---|
| Thymol | 0.01 - 0.1 |
| Undecylenic acid | 0.01 - 0.1 |
| Lactic acid /citric acid | 0.1 - 3 |
| Benzyl alcohol | 0.1 - 3 |
| SDA 3C | 0.1 - 20 |
| Lemongrass oil | 0.01 - 2 |
| Lemon extract | 0.01 - 2 |
| Cocoamidopropyl betaine or Cocoamidopropylamine Oxide (incromine oxide) | 0.2 - 3 |
| Tween 20 | 0.01 - 2 |
| Sodium lauryl sulfate | 0.001 - 2 |
| Solubilizer | 0.001 - 5 |
| Cetrimonium chloride | 0.001 - 5 |
| Zemea (1,3 propanediol) | 0.1 - 2 |
| Water | q.s |
| Total | 100 |

TABLE 2

CRB- BSD-7

| Ingredient | %w/w |
|---|---|
| Thymol | 0.03 |
| Lactic acid | 1 |
| Benzyl alcohol | 1 |
| SDA 3C | 15 |
| Lemongrass oil | 1 |
| Lemon extract | 0.5 |
| Cocoamidopropyl betaine | 1 |
| Tween 20 | 0.5 |
| Solubilizer | 2.5 |
| Zemea (1,3 propanediol) | 0.5 |
| Water | q.s |
| Total | 100 |
| Log reduction | 3 |

TABLE 3

CRB- BSD-7U

| Ingredient | %w/w |
|---|---|
| Undecylenic acid | 0.03 |
| Lactic acid | 1 |
| Benzyl alcohol | 1 |
| SDA 3 C | 15 |
| Lemongrass oil | 1 |
| Lemon extract | 0.5 |
| Cocoamidopropyl betaine | 1 |
| Tween 20 | 0.5 |
| Solubilizer | 2.5 |
| Zemea (1,3 propanediol) | 0.5 |
| Water | q.s |
| Total | 100 |
| Log reduction | 3.5 |

TABLE 4

CRB- BSD-7UT

| Ingredient | %w/w |
|---|---|
| Undecylenic acid | 0.015 |
| Thymol | 0.015 |
| Lactic acid | 1 |
| Benzyl alcohol | 1 |
| SDA 3 C | 15 |
| Lemongrass oil | 1 |
| Lemon extract | 0.5 |
| Cocoamidopropyl betaine | 1 |
| Tween 20 | 0.5 |
| Solubilizer | 2.5 |
| Zemea (1,3 propanediol) | 0.5 |
| Water | q.s |
| Total | 100 |
| Log reduction | 3.7 |

TABLE 5

CRB- BSD-7A

| Ingredient | %w/w |
|---|---|
| Thymol | 0.05 |
| Lactic acid | 1 |
| Benzyl alcohol | 1 |
| SDA 3 C | 15 |
| Lemongrass oil | 1 |
| Lemon extract | 0.5 |
| Cocoamidopropyl betaine | 1 |
| Tween 20 | 0.5 |
| Solubilizer | 2.5 |
| Zemea (1,3 propanediol) | 0.5 |
| Water | q.s |
| Total | 100 |
| Log reduction | 3.5 |

TABLE 6

CRB- BSD-7AS

| Ingredient | %w/w |
|---|---|
| Thymol | 0.05 |
| Lactic acid | 1 |
| Benzyl alcohol | 1 |
| SDA 3 C | 15 |
| Lemongrass oil | 1 |
| Lemon extract | 0.5 |
| Cocoamidopropyl betaine | 1 |
| Tween 20 | 0.5 |
| Sodium lauryl sulfate | 1 |
| Zemea (1,3 propanediol) | 0.5 |
| Water | q.s |
| Total | 100 |
| Log reduction | 3.5 |

TABLE 7

CRB- BSD-8A

| Ingredient | %w/w |
|---|---|
| Lemongrass oil | 1 |
| Lactic acid | 2 |
| Benzyl alcohol | 1 |
| Phenoxy ethanol | 1 |
| SDA 3 C | 15 |
| Glucopon | 0.5 |
| Cocoamidopropyl betaine | 1 |
| Tween20 | 0.5 |
| Solubilizer | 2.5 |
| Zemea (1,3 propanediol) | 0.5 |
| Water | q.s |
| Total | 100 |
| Log reduction | 5.6 |

TABLE 8

CRB- BSD-8

| Ingredient | %w/w |
|---|---|
| Thymol | 0.03 |
| Lactic acid | 1 |
| Benzyl alcohol | 0.5 |
| Phenoxy ethanol | 0.5 |
| SDA 3 C | 15 |
| Cetrimonium chloride(30%) | 4 |
| Cocoamidopropyl betaine | 1 |
| Tween20 | 0.5 |
| Solubilizer | 2.5 |
| Zemea (1,3 propanediol) | 0.5 |
| Water | q.s |
| Total | 100 |
| Log Reduction | 5.1 |

TABLE 9

CRB- BSD-9A

| Ingredient | %w/w |
|---|---|
| Thymol | 0.03 |
| Lactic acid | 2 |
| Benzyl alcohol | 1 |
| SDA 3 C | 15 |
| Phenoxyethanol | 1 |
| Cocoamidopropyl betaine | 1 |
| Tween 20 | 0.5 |
| Octanediol | 0.5 |
| Zemea (1,3 propanediol) | 0.5 |
| Water | q.s. |
| Total | 100 |
| Log reduction | 5.6 |

TABLE 10

CRB- BSD-9A-U

| Ingredient | %w/w |
|---|---|
| Undecylenic acid | 0.03 |
| Lactic acid | 2 |
| Benzyl alcohol | 1 |
| SDA 3 C | 15 |
| Phenoxyethanol | 1 |
| Cocoamidopropyl betaine | 1 |
| Tween 20 | 0.5 |
| Octanediol | 0.5 |
| Zemea (1,3 propanediol) | 0.5 |
| Water | q.s. |
| Total | 100 |
| Log reduction | 5.6 |

TABLE 11

CRB- BSD-9A-UT

| Ingredient | %w/w |
|---|---|
| Thymol | 0.015 |
| Undecylenic acid | 0.015 |
| Lactic acid | 2 |
| Benzyl alcohol | 1 |
| SDA 3 C | 15 |
| PHENOXYETHANOL | 1 |
| Cocoamidopropyl betaine | 1 |
| Tween 20 | 0.5 |
| Octanediol | 0.5 |
| Zemea (1,3 propanediol) | 0.5 |
| Water | q.s. |
| Total | 100 |
| Log reduction | 5.6 |

TABLE 12

CRB- BSD-9A-CC

| Ingredient | %w/w |
|---|---|
| Cetrimonium chloride | 0.015 |
| Lactic acid | 2 |
| Benzyl alcohol | 1 |
| SDA 3 C | 15 |
| Phenoxyethanol | 1 |
| Cocoamidopropyl betaine | 1 |
| Tween 20 | 0.5 |
| Octanediol | 0.5 |
| Zemea (1,3 propanediol) | 0.5 |
| Water | q.s. |
| Total | 100 |
| Log reduction | 5.6 |

TABLE 13

CRB- BSD-9B

| Ingredient | %w/w |
|---|---|
| Thymol | 0.05 |
| Lactic acid | 2 |
| Benzyl alcohol | 1 |
| SDA 3 C | 15 |
| Phenoxyethanol | 1 |
| Cocoamidopropyl betaine | 1 |
| Tween 20 | 0.5 |
| Octanediol | 0.5 |
| Zemea (1,3 propanediol) | 0.5 |
| Water | q.s. |
| Total | 100 |
| Log reduction | 5.6 |

TABLE 14

CRB- BSD-9B-CC

| Ingredient | %w/w |
|---|---|
| Cetrimonium chloride | 0.05 |
| Lactic acid | 2 |
| Benzyl alcohol | 1 |
| SDA 3 C | 15 |
| Phenoxyethanol | 1 |
| Cocoamidopropyl betaine | 1 |
| Tween 20 | 0.5 |
| Octanediol | 0.5 |
| Zemea (1,3 propanediol) | 0.5 |
| Water | q.s. |
| Total | 100 |
| Log reduction | 5.6 |

TABLE 15

CRB-BSD-8AL

| | |
|---|---|
| Lemongrass | 1 |
| Lactic acid | 2 |
| Benzyl alcohol | 1 |
| Phenoxyethanol | 1 |
| SDA-3C | 15 |
| Octanediol | 0.5 |
| Cocoamidopropyl betaine | 1 |
| Tween 20 | 0.5 |
| Solubilizer | 2.5 |
| Zemea (1,3 propanediol) | 0.5 |
| Water | q.s. |
| Total | 100 |
| Log reduction | 5.6 |

TABLE 16

CRB- BSD-9B

| Ingredient | %w/w |
|---|---|
| Thymol | 0.05 |
| Lactic acid | 2 |
| Benzyl alcohol | 1 |
| SDA 3 C | 15 |
| Phenoxyethanol | 1 |
| Cocoamidopropyl betaine | 1 |
| Tween 20 | 0.5 |
| Octanediol | 0.5 |
| Zemea (1,3 propanediol) | 0.5 |
| Water | q.s |
| Total | 100 |
| Log reduction | 5.6 |

TABLE 17

CRB- BSD-9D

| Ingredient | %w/w |
|---|---|
| Thymol | 0.03 |
| Lactic acid | 0.5 |
| Benzyl alcohol | 0.5 |
| SDA 3 C | 15 |
| Cocoamidopropyl betaine | 1 |
| Tween 20 | 0.5 |
| Octanediol | 0.5 |
| Zemea (1,3 propanediol) | 0.5 |
| Water | q.s |
| Total | 100 |
| Log reduction | 4.0 |

TABLE 18

CRB- BSD-8AL-R

| Ingredient | %w/w |
|---|---|
| Lemongrass | 0.5 |
| Lactic acid | 0.5 |
| Orange oil | 0.03 |
| Benzyl alcohol | 0.5 |
| Phenoxyethanol | 0.5 |
| SDA-3C | 10 |
| Octanediol | 0.1 |
| Cocoamidopropyl betaine | 1 |
| Tween 20 | 0.1 |
| Solubilizer | 1 |
| Zemea (1,3 propanediol) | 0.53 |
| Water | q.s. |
| Total | 100 |
| Log reduction | 4.9 |

TABLE 19

CRB- BSD-9C

| Ingredient | %w/w |
|---|---|
| Thymol | 0.03 |
| Lactic acid | 0.5 |
| Benzyl alcohol | 1 |
| SDA 3 C | 15 |
| Phenoxyethanol | 1 |
| Cocoamidopropyl betaine | 1 |
| Tween 20 | 0.5 |
| Octanediol | 0.5 |
| Zemea (1,3 propanediol) | 0.5 |
| Water | q.s |
| Total | 100 |
| Log reduction | 4.91 |

TABLE 20

CRB- BSD-L1

| Ingredient | %w/w |
|---|---|
| Lemongrass | 0.5 |
| Lactic acid | 0.5 |
| Orange oil | 0.03 |
| Benzyl alcohol | 0.25 |
| Phenoxyethanol | 0.25 |
| SDA-3C | 1.12 |
| Octanediol | 0.25 |
| Cocoamidopropyl betaine | 0.9 |
| Tween 20 | 0.1 |
| Solubilizer | 1 |
| Zemea (1,3 propanediol) | 0.6 |
| Water | q.s. |
| Total | 100 |
| Log reduction | 3.64 |

TABLE 21

CRB- BSD-T1

| Ingredient | %w/w |
|---|---|
| Thymol | 0.03 |
| Lactic acid | 0.5 |
| Benzyl alcohol | 0.5 |
| Phenoxyethanol | 0.25 |
| SDA-3C | 2 |
| Phenoxyethanol | 0.5 |
| Cocoamidopropyl betaine | 0.8 |
| Octanediol | 0.3 |
| Zemea (1,3 propanediol) | 0.87 |
| Water | q.s. |
| Total | 100 |
| Log reduction | 4.05 |

TABLE 22

CRB- BSD-L2

| Ingredient | %w/w |
|---|---|
| Lemongrass | 0.5 |
| Lactic acid | 0.5 |
| Orange oil | 0.03 |
| Benzyl alcohol | 0.5 |
| Phenoxyethanol | 0.5 |
| SDA-3C | 1.8 |
| Octanediol | 0.25 |
| Cocoamidopropyl betaine | 0.9 |
| Tween 20 | 0.1 |
| Solubilizer | 1 |
| Zemea (1,3 propanediol) | 0.6 |
| Water | q.s. |
| Total | 100 |
| Log reduction | 3.0 |

TABLE 23

CRB- BSD-T2

| Ingredient | %w/w |
|---|---|
| Thymol | 0.03 |
| Lactic acid | 0.5 |
| Benzyl alcohol | 1 |
| SDA-3C | 2 |
| Phenoxyethanol | 1 |
| Cocoamidopropyl betaine | 0.8 |
| Octanediol | 0.3 |
| Zemea (1,3 propanediol) | 1.1 |
| Water | q.s. |
| Total | 100 |
| Log reduction | 4.61 |

TABLE 24

| CRB- BSD | T3 | T4 |
|---|---|---|
| Ingredient | %w/w | %w/w |
| Thymol | 0.03 | 0.03 |
| Lactic acid | 0.5 | 0.5 |
| Benzyl alcohol | 0.5 | 1 |
| SDA-3C | 2 | 2 |
| Phenoxyethanol | 0.5 | 1 |
| Cocoamidopropyl betaine | 0.8 | 0.8 |
| Octanediol | 0.3 | 0.3 |
| Zemea (1,3 propanediol) | 0.87 | 1.1 |
| Water | q.s. | q.s. |
| Total | 100 | 100 |

TABLE 25

| CRB- BSD | L3 | L4 | L5 |
|---|---|---|---|
| Ingredient | %w/w | %w/w | %w/w |
| Lemongrass | 0.5 | 0.5 | 0.5 |
| Lactic acid | 0.5 | 0.5 | 0.5 |
| Orange oil | 0.03 | 0.03 | 0.03 |
| Benzyl alcohol | 0.25 | 0.5 | 0.5 |
| Phenoxyethanol | 0.25 | 0.5 | 0.5 |
| SDA-3C | 1.12 | 1.8 | 5.6 |
| Octanediol | 0.25 | 0.25 | 0.25 |
| Cocoamidopropyl betaine | 0.9 | 0.9 | 0.9 |
| Tween 20 | 0.1 | 0.1 | 0.1 |
| Solubilizer | 1 | 1 | 1 |
| Zemea (1,3 propanediol) | 0.6 | 0.6 | 0.6 |
| Water | q.s. | q.s. | q.s. |
| Total | 100 | 100 | 100 |
| Log Reduction | 3 | 3 | 3 |

TABLE 26

| CRB- BSD | L3 | L6 |
|---|---|---|
| Ingredient | %w/w | %w/w |
| Lemongrass | 0.5 | 0.5 |
| Lactic acid | 0.5 | 1.5 |
| Orange oil | 0.03 | 0.03 |
| Benzyl alcohol | 0.25 | 0.25 |
| Phenoxyethanol | 0.25 | 0.25 |
| SDA-3C | 1.12 | 1.12 |
| Octanediol | 0.25 | 0.25 |
| Cocoamidopropyl betaine | 0.9 | 0.9 |
| Tween 20 | 0.1 | 0.1 |
| Solubilizer | 1 | 1 |
| Zemea (1,3 propanediol) | 0.6 | 0.6 |
| Water | q.s. | q.s. |
| Total | 100 | 100 |
| Log Reduction | 3.9 | 3.9 |

TABLE 27

| CRB- BSD | L7 | L8 |
|---|---|---|
| Ingredient | %w/w | %w/w |
| Lemongrass | 0.5 | 0.5 |
| Lactic acid | 1.5 | 1.5 |
| Orange oil | 0.03 | 0.03 |
| Benzyl alcohol | 0.5 | 0.5 |
| Phenoxyethanol | 0.5 | 0.5 |
| SDA-3C | 1.12 | 4.62 |
| Octanediol | 0.25 | 0.25 |
| Cocoamidopropyl betaine | 0.9 | 0.9 |
| Tween 20 | 0.1 | 0.1 |
| Solubilizer | 1 | 1 |
| Zemea (1,3 propanediol) | 0.6 | 0.6 |
| Water | q.s. | q.s. |
| Total | 100 | 100 |
| Log Reduction | 3.9 | 3.9 |

TABLE 28

CRB- BSD-L1-Z

| Ingredient | %w/w |
|---|---|
| Lemongrass | 0.5 |
| Lactic acid | 0.5 |
| Orange oil | 0.03 |
| Thymol | 0.01 |
| Undecylenic acid | 0.03 |
| SDA-3C | 1.12 |
| Octanediol | 0.25 |
| Cocoamidopropyl betaine | 0.9 |
| Tween 20 | 0.1 |
| Zemea (1,3 propanediol) | 0.5 |
| Solubilizer | 1 |
| Sodium lauryl sulfate | 1 |
| Water | q.s. |
| Total | 100 |

TABLE 29

CRB- BSD-T1-Z

| Ingredient | %w/w |
|---|---|
| Thymol | 0.03 |
| Lactic acid | 0.5 |
| Undecylenic acid | 0.03 |
| SDA-3C | 2 |
| Sodium lauryl sulfate | 1 |
| Cocoamidopropyl betaine | 0.8 |
| Zemea (1,3 propanediol) | 0.37 |
| Octanediol | 0.3 |
| Water | q.s. |
| Total | 100 |
| Log reduction | 4.05 |

TABLE 30

CRB- BSD-L2-Z

| Ingredient | %w/w |
|---|---|
| Lemongrass | 0.5 |
| Lactic acid | 0.5 |
| Orange oil | 0.03 |
| Cetrimonium chloride | 0.03 |
| SDA-3C | 1.8 |
| Octanediol | 0.25 |
| Cocoamidopropyl betaine | 0.9 |
| Tween 20 | 0.1 |
| Solubilizer | 1 |
| Zemea (1,3 propanediol) | 0.5 |
| Water | q.s. |
| Total | 100 |
| Log reduction | 3 |

TABLE 31

CRB- BSD-T2-Z

| Ingredient | %w/w |
|---|---|
| Thymol | 0.03 |
| Lactic acid | 0.5 |
| Cetrimonium chloride | 0.03 |
| Phenoxyethanol | 1 |
| Cocoamidopropyl betaine | 0.8 |
| Zemea (1,3 propanediol) | 0.6 |
| Octanediol | 0.3 |
| Water | q.s. |
| Total | 100 |
| Log reduction | 4.61 |

TABLE 32

CRB- BSD-T1-UZ

| Ingredient | %w/w |
|---|---|
| Thymol | 0.015 |
| Lactic acid | 0.5 |
| Undecylenic acid | 0.03 |
| SDA-3C | 2 |
| Cocoamidopropyl betaine | 0.8 |
| Zemea (1,3 propanediol) | 0.37 |
| Octanediol | 0.3 |
| Water | q.s. |
| Total | 100 |

TABLE 33

CRB- BSD-L-UZ

| Ingredient | %w/w |
|---|---|
| Lemongrass | 0.35 |
| Lactic acid | 0.5 |
| Orange oil | 0.03 |
| Sodium dodecyl sulfate | 0.2 |
| Undecylenic acid | 0.15 |
| SDA-3C | 1.8 |
| Octanediol | 0.25 |
| Cocoamidopropyl betaine | 0.9 |
| Tween 20 | 0.1 |
| Solubilizer | 1 |
| Zemea (1,3 propanediol) | 0.5 |
| Water | q.s. |
| Total | 100 |

TABLE 34A

CRB- BSD-T2-UZ

| Ingredient | %w/w |
|---|---|
| Undecylenic acid | 0.03 |
| Lactic acid | 0.5 |
| SDA 40 B | 5 |
| Sodium lauryl sulfate | 0.2 |
| Octanediol | 0.3 |
| Zemea (1,3 propanediol) | 1.1 |
| Water | q.s. |
| Total | 100 |

TABLE 34B

CRB- BSD-C-U

| Ingredient | %w/w |
|---|---|
| Undecylenic acid | 0.03 |
| Citric Acid | 0.15 |
| SDA 40 B | 5.0 |
| Sodium lauryl sulfate | 0.03 |
| 1,3 propanediol | 0.05 |
| Octanediol | 0.005 |
| Water | q.s |
| Total | 100 |

### Study B. Surface Disinfectant Formulations for Use as Dilutable Solutions (Concentrate Formulations)

The following formulations are in the concentrate form and should be diluted with water appropriately before use (dilute about 10 to 60 fold).

TABLE 35A

Formulation Ranges (for stock)

| Ingredient | %w/w |
|---|---|
| Thymol | 0.1 -6 |
| Undecylenic acid | 0.1 - 6 |
| Lactic acid | 0.1 -1 |
| Benzyl alcohol | 2-20 |
| SDA 3 C | 10-50 |
| Lemongrass oil | 1 - 5 |
| Lemon extract | 1 - 5 |
| Cocoamidopropyl betaine | 0.001 - 10 |
| Tween 20 | 0.001 - 5 |
| Sodium lauryl sulfate | 2 - 10 |
| Solubilizer | 0.001 - 10 |
| Cetrimonium chloride | 0.001 - 10 |
| Zemea (1,3 propanediol) | 5 - 50 |
| Propylene glycol | 5 - 50 |

TABLE 35B

| CRB-SD-UC (Stock) | % |
|---|---|
| Undecylenic acid | 0.5 |
| Citric acid | 3.0 |
| 1,3 propanediol | 1.2 |
| Octanediol | 0.09 |
| SDS | 0.35 |
| Orange oil | 0.15 |
| SDA 3C alcohol | 4.0 |
| Water | q.s. |
| Total | 100 |
| CRB-SD-UC-30 (1 to 30 dilution with water) | % |
| Undecylenic acid | 0.02 |
| Citric acid | 0.1 |
| 1,3 propanediol | 0.05 |
| Octanediol | 0.003 |
| SDS | 0.012 |
| Orange oil | 0.005 |
| SDA-3C Alcohol | 0.13 |
| Water | q.s. |
| CRB-UTC (Stock) | % |
| Thymol | 0.25 |
| Undecylenic acid | 0.5 |
| Octanediol | 0.09 |
| SDS | 0.35 |
| Orange oil | 0.15 |
| SDA 3C | 4.0 |
| Water | q.s. |
| CRB-UTC-30 (1 to 30 dilution with water) | % |
| Thymol | 0.01 |
| Undecylenic acid | 0.02 |
| Octanediol | 0.003 |
| SDS | 0.015 |
| Orange oil | 0.005 |
| SDA 3C | 0.15 |
| Water | q.s. 100 |
| CRB-BSD-T-1–Stock | |
| Ingredient | %w/w |
| Thymol | 0.6 |
| Lactic acid | 10 |
| Benzyl alcohol | 5 |
| SDA 3 C | 30 |
| PHENOXYETHANOL | 10 |
| Cocoamidopropyl betaine | 16 |
| Tween 20 | 10 |
| Zemea (1,3 propanediol) | 10 |
| Octanediol | 6 |
| Cetrimonium chloride | 0.6 |
| Water | q.s. |

TABLE 36

| | |
|---|---|
| CRB-50-SD Stock | % |
| Thymol | 6 |
| Lactic Acid | 10 |
| Orange oil | 5 |
| Octanediol | 5 |
| phenoxyethanol | 2.5 |
| Benzyl alcohol | 2.5 |
| SDA-3C | 4 |
| Zemea (1,3 propanediol) | 5 |
| Water | q.s. |

TABLE 37

| | |
|---|---|
| CRB-50-SD-2 Stock | % |
| Thymol | 2.08 |
| Lactic Acid | 3.47 |
| Orange oil | 1.73 |
| Octanediol | 1.73 |
| phenoxyethanol | 0.87 |
| Benzyl alcohol | 0.87 |
| SDA-3C | 20 |
| Zemea (1,3 propanediol) | 5 |
| Water | q.s. |

TABLE 38

| | |
|---|---|
| CRB-BSD-L-1-S Stock | % |
| Lemongrass | 10 |
| Lactic Acid | 10 |
| Orange oil | 0.6 |
| Sodium lauryl sulfate | 1 |
| SDA-3C | 22 |
| Octanediol | 5 |
| Cocoamidopropyl betaine | 18 |
| Tween 20 | 2 |
| Zemea (1,3 propanediol) | 10 |
| Solubilizer | 20 |
| Water | q.s. |

TABLE 39

CRB- BSD-T-1-S Stock

| Ingredient | %w/w |
|---|---|
| Thymol | 0.6 |
| Lactic acid | 10 |
| Benzyl alcohol | 5 |
| SDA 3 C | 30 |
| phenoxyethanol | 10 |
| Cocoamidopropyl betaine | 16 |
| Tween 20 | 10 |
| Zemea (1,3 propanediol) | 10 |
| Octanediol | 6 |
| Cetrimonium chloride | 0.6 |
| Water | q.s. |

TABLE 40

CRB- BSD- T-1-S-U Stock

| Ingredient | %w/w |
|---|---|
| Thymol | 0.2 |
| Lactic acid | 10 |
| Undecylenic acid | 0.4 |
| SDA 3 C | 30 |
| Sodium lauryl sulfate | 5 |
| Tween 20 | 10 |
| Zemea (1,3 propanediol) | 10 |
| Octanediol | 6 |
| Water | q.s. |

TABLE 41

CRB- BSD-T-1-30 (1 to 30 dilution with water)

| Ingredient | %w/w |
|---|---|
| Thymol | 0.02 |
| Lactic acid | 0.33 |
| Benzyl alcohol | 0.17 |
| SDA 3 C | 1 |
| phenoxyethanol | 0.33 |
| Cocoamidopropyl betaine | 0.53 |
| Tween 20 | 0.33 |
| Zemea (1,3 propanediol) | 0.33 |
| Octanediol | 0.2 |
| Cetrimonium chloride | 0.02 |
| Water | q.s. |

TABLE 42

CRB- BSD- T-1-S-U-Stock

| Ingredient | %w/w |
|---|---|
| Thymol | 0.2 |
| Lactic acid | 10 |
| Undecylenic acid | 0.4 |
| SDA 3 C | 30 |
| Sodium lauryl sulfate | 5 |
| Tween 20 | 10 |
| Zemea (1,3 propanediol) | 10 |
| Octanediol | 6 |
| Water | q.s. |

TABLE 43

CRB- BSD- T-1-S-U-30 (1 to 30 dilution with water)

| | |
|---|---|
| Ingredient | %w/w |
| Thymol | 0.01 |
| Lactic acid | 0.33 |
| Undecylenic acid | 0.01 |
| SDA 3 C | 1 |
| Sodium lauryl sulfate | 0.17 |
| Tween 20 | 0.33 |
| Zemea (1,3 propanediol) | 0.33 |
| Octanediol | 0.2 |
| Water | q.s. |

TABLE 43A

CRB- BSD- T-1-S-UP-Stock

| | |
|---|---|
| Ingredient | %w/w |
| Thymol | 0.2 |
| Lactic acid | 10 |
| Undecylenic acid | 0.4 |
| SDA 3 C | 30 |
| Sodium lauryl sulfate | 5 |
| Tween 20 | 10 |
| Propylene Glycol | 10 |
| Octanediol | 6 |
| Water | q.s. |

TABLE 43B

CRB- BSD- T-1-S-UP-30 (1 to 30 dilution with water)

| | |
|---|---|
| Ingredient | %w/w |
| Thymol | 0.01 |
| Lactic acid | 0.33 |
| Undecylenic acid | 0.01 |
| SDA 3 C | 1 |
| Sodium lauryl sulfate | 0.17 |
| Tween 20 | 0.33 |
| Propylene glycol | 0.33 |
| Octanediol | 0.2 |
| Water | q.s. |

Study 3: Optimized Direct Use Surface Disinfectant Formulations (Spray or Wipes)

TABLE 44

Main Ingredient Ranges

| Ingredient | %w/w |
|---|---|
| Thymol | 0.001 - 0.1 |
| Undecylenic acid | 0.001 - 0.1 |
| Lactic acid /citric acid | 0.5-3 |
| Denatured alcohol | 0.1 - 4 |
| Cocoamidopropyl betaine | 0.2 - 3 |
| Cetrimonium chloride | 0.001 - 5 |
| Sodium dodecyl sulfate | 0.001 - 2 |
| Orange oil | 0.01 - 0.1 |
| Incromine oxide | 0.001 - 2 |
| 1,3-propanediol (Zemea) | 0.01 - 1 |
| Propylene glycol | 0.01 - 1 |
| Water | q.s |
| Total | 100 |

TABLE 45

CRB-BSD-E1 Sock solution (Dilution of 1 to 30 -1 to 60 with water before use)

| | |
|---|---|
| Ingredient | % w/w |
| Undecylenic acid | 1.5 |
| Thymol | 0 |
| Citric acid | 20 |
| Sodium dodecyl sulfate | 2 |
| Denatured alcohol | 50 |
| Cocamide propyl betaine | 3 |
| Incromine oxide | 2 |
| Water | q.s. |

Formulations and antibacterial efficacy (Log reduction) by EN 1276 method against S. aureus.

TABLE 46

CRB-BSD-E1

| Ingredient | %w/w |
|---|---|
| Undecylenic acid | 0.05 |
| Citric acid | 1 |
| Sodium dodecyl sulfate | 0.5 |
| Denatured alcohol | 30 |
| Cocamide propyl betaine | 1 |
| Incromine oxide | 1 |
| Water | q.s |
| Log reduction | 5.3 |

TABLE 47

CRB-BSD-E2 Stock solution (Dilute 1 to 30 -1 to 60 with water before use)

| | |
|---|---|
| Ingredient | % w/w |
| Thymol | 1.5 |
| Citric acid | 20 |
| Sodium dodecyl sulfate | 2 |
| Denatured alcohol | 50 |
| Cocamide propyl betaine | 3 |
| Incromine oxide | 2 |
| Water | q.s. |

TABLE 48

CRB-BSD-E2

| Ingredient | % w/w |
|---|---|
| Thymol | 0.05 |
| Citric acid | 1 |
| Sodium dodecyl sulfate | 0.5 |
| Denatured alcohol | 30 |
| Cocamide propyl betaine | 1 |
| Incromine oxide | 1 |
| Water | q.s |
| Log reduction | 5.2 |

TABLE 49A

CRB-BSD-E3 (Direct formulation, not from concentrate)

| Ingredient | % w/w |
|---|---|
| Undecylenic acid | 0.02 |
| Thymol | 0.05 |
| Citric acid | 1 |
| Orange oil | 0.01 |
| Cetrimonium chloride | 0.03 |
| Denatured alcohol | 30 |
| Cocamide propyl betaine | 1 |
| Incromine oxide | 1 |
| Water | q.s |
| Log reduction | 5.5 |

TABLE 49B

CRB-BSD-E4

| Ingredient | % w/w |
|---|---|
| Undecylenic acid | 0.02 |
| Thymol | 0.02 |
| Citric acid | 1 |
| Cetrimonium chloride | 0.03 |
| Orange oil | 0.01 |
| Denatured alcohol | 30 |
| Cocamide propyl betaine | 1 |
| Incromine oxide Sodium citrate | 1 0.2 |
| Water | q.s |
| Log reduction | 5.5 |

TABLE 49C

CRB-BSD-E4 Stock solution (for 1 to 10 dilution)

| | |
|---|---|
| Ingredient | % w/w |
| Undecylenic acid | 0.2 |
| Thymol | 0.2 |
| Citric acid | 10 |
| Cetrimonium chloride | 0.3 |
| Orange oil | 0.1 |
| Denatured alcohol | 50 |
| Cocamide propyl betaine | 5 |
| Incromine oxide | 5 |
| Sodium citrate | 2 |
| Water | q.s |

TABLE 50

CRB-BSD-F1 Stock solution (Dilute 1 to 30 -1 to 60 with water before use)

| Ingredient | % w/w |
|---|---|
| Undecylenic acid | 1.5 |
| Thymol | 1.5 |
| Citric acid | 20 |
| Sodium dodecyl sulfate | 2 |
| Denatured alcohol | 50 |
| Cocamide propyl betaine | 3 |
| Incromine oxide | 2 |
| Water | 20 |

TABLE 51

CRB-BSD-F1

| Ingredient | % w/w |
|---|---|
| Undecylenic acid | 0.02 |
| Thymol | 0.02 |
| Citric acid | 1 |
| Sodium dodecyl sulfate | 0.5 |
| Denatured alcohol | 30 |
| Cocamide propyl betaine | 1 |
| Incromine oxide | 1 |
| Water | q.s |
| Log reduction | 5.5 |

### Study 4: Antimicrobial Efficacy Study of CRB-BSD-F1 Surface Disinfectant Formulation Using the EPA 13 697 Disk Method (S. aureus)

Method: Stainless steel disks of diameter 2.0 cm were sterilized using alcohol and allowed to dry completely. Equal parts of 3×10⁸ CFU/mL of test pathogen and 3 g/L bovine serum albumin were mixed prior to inoculation. 50 µL of pathogen-bovine mixture was plated on the sterile disk and incubated for 30 minutes. Once all the liquid has evaporated, 100 µL of test surface disinfectant solution and PBS for control was spread on the plate and allowed contact for 5 minutes. After 5 minutes, the disk was carefully transferred onto D/E neutralizing media. The groups were then diluted as appropriate and plated. Lysol® surface disinfectant was tested for comparison. Two (2) surface disinfectants CRB-BSD-F1 and CRB-BSD- E4 were tested, and results showed that the log reduction for these were 3.5, while that for Lysol® is 2.5 from control growth EPA 13 697.

### Study 5: In Vitro Rapid Kill Study Using the ASTM E2783-11 Method

Method: In vitro rapid-kill test was carried out according to ASTM E2783-11; 10⁸ CFU mL⁻¹ microbial cultures were prepared in a TSA media in order to determine the antimicrobial activity of surface disinfectants. 0.9 mL surface disinfectants were kept in contact with 0.1 mL various microbial cultures for 15 seconds and subsequently neutralized using agent DE. PBS was used as a control. The neutralized cultures were appropriately diluted and plated on agar plates. Colony count was determined after overnight incubation at 37° C.

Table 51A: Results for in vitro rapid kill study using the ASTM E2783-11 method

TABLE 51A

Log Reduction

| Group | S. aureus | MRSA | E. Coli | P. eruginosa | M. smegmatis | S. typhimurium |
|---|---|---|---|---|---|---|
| CRB-BSD-F1 | 5.0 | 5.0 | 5.0 | 5.0 | 5.0 | 5.0 |
| Seventh Generation® | 4.0 | 3.0 | 5.0 | 3.0 | 1.0 | 5.0 |
| Lysol® | 5.0 | 5.0 | 5.0 | 4.0 | 3.0 | 5.0 |
| Proquat® | 4.0 | 4.0 | 3.0 | 4.0 | 5.0 | 4.0 |

The following surface disinfectants were prepared and tested for antibacterial efficacy by ASTM E2197 Method after 1 to 10 dilution, using S. aureus as the test organism.

### Quantitative Disk Carrier Test ASTM E2197

The ASTM E2197 method is designed to test bactericidal, fungicidal, sporicidal, and virucidal capabilities of liquid germicides on non-porous hard surfaces using a 1 cm diameter brushed stainless steel surface.
50 µL of test pathogen is added to the center of 1 cm diameter brushed stainless steel disks.
The test pathogen is dried at 37° C. for 1 hour.
After drying, the carriers are individually placed into the bottom of sterile vessels for testing.
100 µL of test substance is added directly to the center of the carrier (on top of the dried inoculum) and allowed to sit undisturbed for the duration of the exposure period (5 mins)
A 10 mL volume of DE neutralizer media is added to each vessel and vortexed to elute surviving test microorganisms.
The resulting eluent is serially diluted, plated in TSA plates and incubated overnight at 37° C.
Colonies are counted and log₁₀ reduction from control is calculated.

Groups tested are shown as follows:

TABLE 51B

Surface disinfectant (SD-60)

| Ingredient | % w/w |
|---|---|
| Undecylenic acid | 2 |
| Sodium citrate | 2 |
| Citric acid | 15 |
| Benzoic acid | 2 |
| SDS | 3 |
| Alcohol 3C | 15 |
| Propylene glycol | 10 |
| Water | 51 |
| Total | 100 |

TABLE 51C

| Ingredient | SD 62O -1 |
|---|---|
| Undecylenic acid | 2 |
| Citric acid | 15 |
| SDA 40B | 15 |
| Benzoic acid | 2 |
| SDS | 3 |
| Sodium Citrate | 3 |
| Propylene Glycol | 10 |
| Octanediol | 0.5 |
| Water (qs to 100) | 49.5 |
| Total | 100 |

TABLE 51D

| Ingredient | SD 62O-P |
|---|---|
| Undecylenic acid | 2 |
| Citric acid | 15 |
| SDA 3C | 15 |
| Benzoic acid | 2 |
| SDS | 3 |
| Sodium Citrate | 2 |
| Propylene Glycol | 10 |
| Octanediol | 0.5 |
| Water (qs to 100) | 50.5 |
| Total | 100 |

TABLE 51E

| Ingredient | SD 62O-Z |
|---|---|
| Undecylenic acid | 2 |
| Citric acid | 15 |
| SDA 3C | 15 |
| Benzoic acid | 2 |
| SDS | 3 |
| Sodium Citrate | 2 |
| 1,3 propanediol (Zemea®) | 10 |
| Octanediol | 0.5 |
| Water (qs to 100) | 50.5 |
| Total | 100 |

TABLE 51F

| Ingredient | SD 60O-P |
|---|---|
| Undecylenic acid | 2 |
| Citric acid | 15 |
| SDA 3C | 10 |
| Benzoic acid | 2 |
| SDS | 3 |
| Sodium Citrate | 2 |
| Propylene Glycol | 10 |
| Octanediol | 0.5 |
| Water (qs to 100) | 55.5 |
| Total | 100 |

TABLE 51G

| Ingredient | SD (60O-Z) |
|---|---|
| Undecylenic acid | 2 |
| Citric acid | 15 |
| SDA 3C | 10 |
| Benzoic acid | 2 |
| SDS | 2 |
| Sodium Citrate | 2 |
| Zemea | 10 |
| Octanediol | 0.5 |
| Water (qs to 100) | 55.5 |
| Total | 100 |

All of the formulations showed more than 5 log reduction of growth from control growth against S. aureus.

### Example 2

### Preservative Formulations and Efficacy

Preservative compositions were prepared, comprising the following ingredients:
(a) 10 to 50% undecylenic acid or 0.02 to 20% thymol, or combinations thereof;
(b) 1 to 80% propanediol; and
(c) 0.1 to 20% organic acid selected from benzoic, citric, lactic, salicylic acid or any combination thereof.

At typical use levels of 1% for preservation of cosmetics and other commercial products, the concentration of these compositions becomes:
(a) 0.1 to 0.5% undecylenic acid or 0.0002 to 0.2% thymol, or combinations thereof;
(b) 0.01 to 0.8% 1,3 propanediol; and
(c) 0.001 to 0.2% organic acid selected from benzoic, citric, lactic, salicylic acid or any combination thereof.

In other embodiments, the compositions comprised one or more of the following:
(d) alkanediol selected from pentylene glycol, octanediol or decanediol;
(e) solvents selected from ethanol, isopropanol, benzyl alcohol, phenoxyethanol, phenyl ethanol; or
(f) essential oils or extracts selected from bergamot oil, myrrh oil, cinnamon leaf oil, cinnamon oil, cinnamon bark oil, lemon oil, lemon extract, lemongrass oil, pomegranate oil, pomegranate extract, grapefruit seed extract, orange oil, cedarwood oil, geranium oil, willow bark extract, willow bark oil, wasabi extract or any combination thereof.

Exemplary formulations were prepared as follows:

TABLE 52

| Ingredients | CP-M-H2 |
|---|---|
| Pentylene glycol | 10 |
| Benzoic Acid | 10 |
| Undecylenic Acid | 30 |
| Zemea (1,3 propanediol) | 49.8 |
| Cinnamon Leaf Oil | 0.2 |
| Total | 100 |

TABLE 53

| Ingredients | CP-9N-NT2 | Actual % |
|---|---|---|
| Pentylene glycol | 50 | 41.7 |
| Benzoic Acid | 10 | 8.3 |
| Undecylenic Acid | 30 | 25 |
| Zemea (1,3 propanediol) | 30 | 25 |
| Total | 120 | 100 |

TABLE 54

| Ingredients | CP-9N-NT | Actual % |
|---|---|---|
| Pentylene glycol | 50 | 41.6 |
| Benzoic Acid | 10 | 8.3 |
| Undecylenic Acid | 30 | 25 |
| Zemea (1,3 propanediol) | 30 | 25 |
| Thymol | 0.2 | 0.2 |
| Total | 120.2 | 100 |

TABLE 55

| Ingredients | CP-M-AH |
|---|---|
| Pentylene glycol | 2 |
| Benzoic Acid | 10 |
| Undecylenic Acid | 30 |
| Zemea (1,3 propanediol) | 58 |
| Total | 100 |

TABLE 56

| Ingredients | CP-M-A1 |
|---|---|
| Benzoic Acid | 10 |
| Undecylenic Acid | 30 |
| Zemea (1,3 propanediol) | 50 |
| Willow bark extract | 10 |
| Total | 100 |

TABLE 57

| Ingredients | CP-M-A2 | Actual % |
|---|---|---|
| Benzoic Acid | 10 | 9.1 |
| Undecylenic Acid | 30 | 27.3 |
| Zemea (1,3 propanediol) | 50 | 45.5 |
| Myrrh oil | 20 | 18.2 |
| Total | 110 | 100 |

TABLE 58

| Ingredients | CP-M-A3 |
|---|---|
| Benzoic Acid | 10 |
| Undecylenic Acid | 30 |
| Zemea (1,3 propanediol) | 50 |
| Wasabi Extract | 10 |
| Total | 100 |

TABLE 59

| Ingredients | CP-M-1A |
|---|---|
| Benzoic Acid | 10 |
| Undecylenic Acid | 30 |
| Zemea (1,3 propanediol) | 60 |
| Thymol | 0.2 |
| Total | 100.2 |

TABLE 60

| Ingredients | CP-9N | Actual % |
|---|---|---|
| Pentylene glycol | 50 | 38.5 |
| Zemea (1,3 propanediol) | 30 | 23.1 |
| Benzoic Acid | 10 | 7.7 |
| Thymol | 10 | 7.7 |
| Undecylenic Acid | 30 | 23.1 |
| Total | 130 | 100 |

Ingredient ranges are shown in Tables 61 through 63 below:

TABLE 61

| Ingredients | Group 1 (as added) |
|---|---|
| Pentylene glycol | 10-50 |
| Zemea (1,3 propanediol) | 30 - 80 |
| Benzoic Acid | 5 - 20 |
| Undecylenic acid | 20-50 |
| Total | 100 - 130 |

TABLE 62

| Ingredients | Group 2 (as added) |
|---|---|
| Pentylene glycol | 10-50 |
| Zemea (1,3 propanediol) | 30 - 80 |
| Benzoic Acid | 5-20 |
| Undecylenic acid | 20-50 |
| Thymol | 1 - 10 |
| Total | 100 - 130 |

TABLE 63

| Ingredients | Group 3 (as added) |
|---|---|
| Pentylene glycol | 10-50 |
| Zemea (1,3 propanediol) | 30-80 |
| Benzoic Acid | 5-20 |
| Undecylenic acid | 20-50 |
| Myrrh oil | 1-10 |
| Total | 100 - 130 |

Optimized formulation is shown below.

TABLE 64

| Ingredients | Group 4 (as added) |
|---|---|
| Zemea (1,3 propanediol) | 30-80 |
| Benzoic Acid | 5-20 |
| Undecylenic acid | 20-50 |
| Thymol | 0.2-1 |
| Total | 100 - 130 |

TABLE 65

| Ingredients | Group 4 PBU |
|---|---|
| Zemea (1,3 propanediol) | 59.5 |
| Benzoic Acid | 10 |
| Undecylenic acid | 30 |
| Thymol | 0.5 |
| Total | 100 - 130 |

TABLE 66

| Ingredients | Group 5 BBU |
|---|---|
| Benzyl alcohol | 59.5 |
| Benzoic Acid | 10 |
| Undecylenic acid | 30 |
| Thymol | 0.5 |
| Total | 100 - 130 |

In certain embodiments, thymol is added as odor masking agent. Other odor masking agents Such as Myrrh oil, orange oil, bergamot oil, cedarwood oil, cinnamon bark oil or essential oil based fragrances can be used from 0.1 to 0.5%.

### Mixing and Testing of Preservative Formulations

1 to 1.7% of a composition herein is added to 25 g of personal care product, such as hydrophilic cream, lotion, or shampoo. 25 mL portions of the resulting compositions are then inoculated with 0.1 mL microorganism suspension (initial microorganism count of approximately 10⁸ cfu/mL) and then stirred. The following important organisms were tested:
Test organisms ATCC -
Aspergillus brasiliensis 16404
Candida albicans 10231
Staphylococcus aureus 6538
The cultures are incubated for 18 hours at 37° C., and colonies counted.
Results are shown in Table 67.

TABLE 67

| Group | C. albicans | A. niger | E. Coli | S. aureus |
|---|---|---|---|---|
| CP-9N | 5.6 | 5.6 | 5.6 | 5.6 |
| CP-9N-NT | 5.6 | 5.6 | 5.6 | 5.6 |
| CP-M-1A | n.d. | n.d. | 5.6 | 5.6 |
| CP-M-A-1 | 5.6 | 5.6 | 5.6 | 5.6 |
| CP-M-A-2 | 5.6 | 5.6 | 5.6 | 5.6 |
| CP-M-A-3 | 5.6 | 5.6 | 5.6 | 5.6 |
| Group 4 PBU | 5.6 | 5.6 | 5.6 | 5.6 |

Example 3: Minimum inhibitory concentration (MIC)

TABLE 68

| Microorganism (ATCC#) | MIC (ppm) Group 4 PBU |
|---|---|
| S. aureus (6538) | 15 |
| E. coli (35218) | 125 |
| P. aeruginosa (9027) | 125 |
| A. niger (16404) | 62.5 |
| C. albicans (10231) | 62.5 |

Example 4: Efficacy test of the Botanical Preservative in a Lotion - CTFA Challenge Test
Group A: The Botanical Preservative was added to several 25 g portions of a hydrophilic lotion and inoculated with 0.1 mL microorganism suspension containing approximately 10⁸ cfu/mL (for A. niger 10⁷ cfu/mL was used) and stirred.
Group B: The same procedure as for Group A was followed except the lotions containing the Botanical Preservative were kept at room temperature for 3 months and then inoculated with the micro-organisms as above.
Group C: The same procedure as for Group A was followed except the Botanical Preservative was kept at 40° C. for 2 weeks (accelerated aging), incorporated into the lotion and then inoculated with the micro-organisms as above.

Table 69. Group A efficacy of the botanical preservative (PBU)

TABLE 69

Group A (Colony counts (CFU)ml)

| | S. aureus ATCC 6538 | P. aeruginosa ATCC 15442 | C. albicans ATCC 10231 | A. niger ATCC 16404 | E. coil ATCC 35218 |
|---|---|---|---|---|---|
| Lotion (Control) | 5×10⁶ | 5×10⁵ | 5×10⁶ | 1×10⁵ | 5×10⁵ |
| 2 day | <10 | <10 | <10 | Not Done | <10 |
| 7 day | <10 | < 1-0 | <10 | <10 | < 10 |
| 14 day | <10 | <10 | <10 | <10 | <10 |
| 21 day | <10 | <10 | <10 | <10 | <10 |
| 28 day | <10 | <10 | <10 | <10 | <10 |

Table 70. Group B efficacy of the botanical preservative in lotion that had been incubated for 3 months before testing.

TABLE 70

Group B(Colony counts (CFU)/ml)

| | S. aureus ATCC 6538 | P. aeruginosa ATCC 15442 | C. albicans ATCC 10231 | A. niger ATCC 16404 | E. coli ATCC 35218 |
|---|---|---|---|---|---|
| Lotion (Control) | 5×10⁶ | 5×10⁶ 5×10⁶ | 5×10⁵ | 1×10⁶ | 5×10⁶ |
| 2 day | <10 | <10 | <10 | Not Done | <:10 |
| 7 day | <10 | <10 | < 10 | <10 | <10 |
| 14 day | <10 | <10 | <10 | <10 | <10 |
| 21 day | <10 | <10 | <10 | <1 0 | <10 |
| 28 day | <10 | <10 | <10 | <10 | <10 |

Table 71. Group C stability and efficacy of the botanical preservative after 2 weeks of accelerated aging then added to the lotion.

TABLE 71

Group C Colony counts (cfu/ml)

| | S. aureus ATCC 6538 | P. aeruginosa ATCC 15442 | C, albicans ATCC 10231 | A. niger ATCC 16404 | E. coli ATCC 35218 |
|---|---|---|---|---|---|
| Lotion (Control) | 5×10⁶ | 5×10⁶ | 5×10⁶ | 1×10⁶ | 5×10⁶ |
| 2 day | <10 | <10 | 1.2×10⁷ | Not Done | <10 |
| 7 day | <10 | <10 | <10 | <10 | <10 |
| 14 day | <10 | <10 | <10 | <10 | <10 |
| 21 day | <10 | <10 | <10 | <10 | <10 |
| 28 day | <10 | <10 | <10 | <10 | <10 |

Table 72: Stable Preservative Formulations and Efficacy

TABLE 72

BNP preservative formulation (General formula)

| | pH 3-3.5 |
|---|---|
| | % |
| Benzoic Acid | 10-20 |
| Undecylenic Acid | 30 - 50 |
| Zemea (1,3 propanediol) | 30 - 50 |
| Essential oils | 0.1 - 1 |
| BNP Preservative 6 CB | % |
| Benzoic Acid | 10 |
| Undecylenic Acid Zemea (1,3 propanediol) | 45 45 |
| CBO | 0.2 |
| Total | 100.2 |
| BNP Preservative 6 LG | % |
| Benzoic Acid | 10 |
| Undecylenic Acid | 45 |
| Zemea (1,3 propanediol) | 45 |
| Lemongrass oil | 0.4 |
| Total | 100.4 |
| BNP Preservative 7 OR | % |
| Benzoic Acid | 10 |
| Undecylenic Acid | 45 |
| Zemea (1,3 propanediol) | 45 |
| Orange oil | 0.6 |
| | pH |
| BNP Preservative 8 LG | % |
| Benzoic Acid | 10 |
| Undecylenic Acid | 45 |
| Zemea (1,3 propanediol) | 45 |
| Lemongrass oil | |
| BNP Preservative 8 LG | % |
| Benzoic Acid | 10 |
| Undecylenic Acid | 45 |
| Zemea (1,3 propanediol) | 45 |
| Lemongrass oil | 1 |
| BNP 9 LG | |
| Benzoic Acid | 10 |
| Undecylenic Acid | 50 |
| Zemea (1,3 propanediol) | 40 |
| Lemongrass oil | 1 |
| BNP Preservative 10 LG | % |
| Benzoic Acid | 15 |
| Undecylenic Acid | 50 |
| Zemea (1,3 propanediol) | 35 |
| Lemongrass oil | 1 |
| BNP Preservative 11 OR | % |
| Benzoic Acid | 10 |
| Undecylenic Acid | 50 |
| Zemea (1,3 propanediol) | 40 |
| Orange oil | 0.3 |

### Antimicrobial Efficacy Testing of Formulations

Preservative formulations were incorporated into a cosmetic cream base at concentration of 1%. Pathogens were added to the cream on Day 0 and subcultured periodically. The following cultures were added into 25 mL of preservative containing cream.
S. aureus: 3 x 10⁷ CFU
P. aeruginosa: 3 x 10⁷ CFU
Aspergillus niger: 3 x 10⁷ CFU
Candida albicans: 3 x 10⁷ CFU

For all of the above formulations, more than 5.0 log reduction against all the bacteria tested was obtained for the study duration of 30 days.

The Botanical Preservative passed all the CTFA preservative tests. It is odorless and colorless and can be used in personal care products ranging from pH 3-8.

Although the present invention has been described in relation to embodiments thereof, these embodiments and examples are merely exemplary and not intended to be limiting. Many other variations and modifications and other uses will become apparent to those skilled in the art. The present invention should, therefore, not be limited by the specific disclosure herein, and can be embodied in other forms not explicitly described here, without departing from the spirit thereof.

1. A botanical antimicrobial composition comprising:
(a) 0.01 to 2% undecylenic acid; or 0.01 to 1% thymol; or a combination thereof;
(b) 1 to 80% of one or more solvents selected from ethanol, propanediol or a combination thereof; and
(c) 0.1 to 20% organic acid, wherein the organic acid is benzoic acid, citric acid, lactic acid, salicylic acid, mandelic acid, maleic acid or any combination thereof.

2. The composition of claim 1, further comprising one or more of the following:
(d) 0.1 to 20% solvent or emollient solvent selected from glycerin, propylene glycol, 1,3 propanediol, benzyl alcohol, phenoxyethanol, phenylethanol, isopropanol, or any combination thereof;
(e) 1 to 20% alkanediol selected from pentylene glycol, octanediol, or decanediol;
(f) one or more essential oils or extracts selected from wasabi extract, cinnamon bark oil, willow bark oil, lemongrass oil, lemon oil, orange oil, cinnamon oil, cedarwood oil, myrrh oil, geranium oil, bergamot oil, or any combination thereof; or
(g) 0.01 to 5% surfactant or chelating agent selected from sodium dodecyl sulfate, Cocoamidopropyl betaine, Incromine oxide, cetrimonium chloride, EDTA, or any combination thereof.

3. The composition of claim 1, wherein the composition exhibits 3.0 or more log₁₀ reduction of bacteria within 5 minutes of contact compared to control.

4. A surface disinfecting or sanitizing composition comprising the antimicrobial composition of claim 1.

5. A preservative composition comprising:
(a) 30 to 50% undecylenic acid; or 1 to 5% thymol, or a combination thereof;
(b) 5 to 50% of one or more solvents selected from ethanol, propanediol, or any combination thereof; and
(c) 10 to 20% organic acid, wherein the organic acid is benzoic acid, citric acid, lactic acid, salicylic acid, mandelic acid, maleic acid, or any combination thereof.

6. The composition of claim 5, further comprising one or more essential oils or extracts selected from wasabi extract, cinnamon bark oil, willow bark oil, lemongrass oil, lemon oil, orange oil, cinnamon oil, cedarwood oil, myrrh oil, geranium oil, bergamot oil or any combination thereof.

7. The composition of claim 5, further comprising 0.01 to 1% of one or more chelating agents selected from sodium phytate, ethylenediaminetetraacetic acid (EDTA), or a combination thereof.

8. A preservative composition comprising 1 to 2% of the composition of claim 5, in a personal care product carrier.

9. A food or personal care preservative composition comprising an antimicrobial composition of claims 1 or 5.

10. A method of preserving food or a personal care composition, the method comprising contacting the food or personal care composition with a composition of claims 1 or 5.
